(19) United States
(12) Patent Application Publication
(10) Pub. No.: US 2019/0374705 A1
(43) Pub. Date: Dec. 12, 2019

(54) Device and Method Useful in Mobile Administering of Feed Products to Patients

(71) Applicant: Staley House LLC, Helena, AR (US)

(72) Inventors: Misti Staley, Helena, AR (US); William Staley, Helena, AR (US)

(21) Appl. No.: 16/427,880

(22) Filed: May 31, 2019

Related U.S. Application Data
(60) Provisional application No. 62/681,911, filed on Jun. 7, 2018.
(60) Provisional application No. 62/764,926, filed on Aug. 16, 2018.
(60) Provisional application No. 62/819,164, filed on Mar. 15, 2019.

(51) Int. Cl. A61M 5/14; A61J 15/00; F16M 13/02; F16B 2/22; F16B 2/06

(52) U.S. Cl. CPC A61M 5/1417; A61J 15/0026; F16M 13/022; F16M 2200/06; F16B 2/065; A61M 2209/082; F16B 2/22

(57) ABSTRACT

A device useful in the administration of feed product from a feeding assembly to a patient, the device having a mounting piece operable to mount the device to a fixed structure, a holding piece operable to hold the feeding assembly, and a flexible arm extending between the mounting piece and locking piece, the flexible arm having sufficient flexibility to be bent such that the feeding assembly can be moved to a desired height but having sufficient rigidity to hold the feeding assembly at the desired height without unintended movement.

### CROSS REFERENCE TO RELATED APPLICATIONS

This application claims priority from (a) U.S. provisional patent application no. 62/681,911 filed on Jun. 7, 2018, and entitled “FreeArm Bolus Feeding Assistant Assembly and Method of Use”, (b) U.S. provisional patent application no. 62/764,926 filed on Aug. 16. 2018, and entitled “Freearm assembly and method of use for mobile medical applications”, and (c) U.S. provisional patent application No. 62/1819,164 filed on Mar. 15, 2019, and entitled “FreeArm Tube Feeding Assistant.” Such applications are incorporated by reference herein in their entirety.

### STATEMENT REGARDING FEDERALLY SPONSORED RESEARCH OR DEVELOPMENT

Not applicable.

### BACKGROUND OF THE INVENTION

Bolus feeding is a method of enteral tube feeding utilizing a syringe, gravity feeding bag, or drip bottle to deliver medication, food, or other compounds. The administration of the product is generally through a series of smaller volume feeds that occur at particular intervals of time. The amount of product that is administered and the particular intervals at which administration occurs depends on the particular needs of the patient. The method is commonly referred to as “gravity feeding” because the syringe, bag, or bottle is typically held at an upward position to allow the product to flow down using gravity. Of course, the positioning of the syringe, bag, or bottle at different heights above the patient affects the feed rate of the bolus device. For this reason, the appropriate positioning of the device is important to patient health and safety. Furthermore, because one of the primary advantages of the bolus feeding method is the flexibility it affords patients and their caregivers between the feeding times, mobility is also an important consideration for bolus feeding apparatuses. Still, given the vitality of the positioning of the feeding apparatus, ease of mounting the device cannot be sacrificed for improved mobility.

With both ease of mounting and mobility in mind, many different techniques for mounting and holding bolus feeding devices have been created and used. In hospital settings across the U.S. bolus feeding is unregulated and there are a number of different ways in which bolus feeding occurs. Some examples of methods that are often used include (a) tying a ribbon across a crib to hang the syringe, bag, or bottle (n cases where the patient is an infant), (b) taping the device to the wall, crib, or bed, (c) taping the device to the shirt of the caregiver who holds the patient during feeding, (d) securing the device with a rubber band to some location on a wall (such as an oxygen meter), and (e) securing the device with a rubber band and tongue depressors and hanging the device through a hole in isolate. While all of these methods technically work to allow feeding through the device, the lack of uniformity (as the method used is generally up to nurse preference) from one instant to another creates substantial risk for hospitals as the chance for human error increases. Furthermore, each of these techniques is lacking in one or both of ease of mounting and mobility. Given the medical nature of such devices and the extreme care that must be used when administering product through such devices, an improved apparatus for securing and mounting bolus feed devices is needed. Furthermore, a device that allows for the regulated and uniform administration of bolus feeding is needed.

### BRIEF SUMMARY OF THE INVENTION

Generally speaking, the present invention is an apparatus for securing and mounting bolus feeding devices and also works as a travel holder for a feeding pump and feed bag, and more particularly to a device having a flexible arm (or gooseneck), clip, and clamp assembly useful for securely holding and mounting bolus feeds to a. variety of surfaces, the apparatus being flexible as to allow the user to adjust the positioning of the bolus feed device to modify feed rates to the appropriate level while maintaining a secure hands-free mounting position. It is a further object of the present invention to provide an apparatus that is capable of being used in hospital settings, home settings, or other settings. It is yet another object of the present invention to provide a device that allows for the regulated and uniform administration of bolus feeding in order to provide a single method of bolus feeding that nurses and physicians at hospitals can learn and perfect, thus limiting risk of error during administration. These and other objects, features, and advantages of the present invention will become better understood from a consideration of the following detailed description of the preferred embodiments and appended claims in conjunction with the drawings as described following:

### BRIEF DESCRIPTION OF THE DRAWINGS

FIG. 1 shows one embodiment of the device of the present invention, wherein the feeding assembly is a bolus syringe feeding assembly.

FIG. 2 shows another embodiment of the device of the present invention wherein the feeding assembly is a bolus syringe feeding assembly.

FIG. 3 shows one embodiment of the assembly of the present invention, wherein the feeding assembly is a bag and pump combination.

FIG. 4 shows another embodiment of the assembly of the present invention, wherein the feeding assembly is a bag and pump combination.

### DETAILED DESCRIPTION OF THE INVENTION

Generally speaking, the present invention is directed to a device 1 useful for securely mounting a feeding assembly (for example, a bolus feeding tube syringe 5 or a feedbag 12 and pump 14 combination) at an adjustable feeding position to a fixed surface or fixed point 6 such as a crib, table, or other structure, and a method for using such a mounting assembly. For purposes of describing the invention, the term “feeding assembly” may be used, it is understood that such term encompasses the bolus feeding tube syringe 5, the feedbag 12 and pump 14 combination, or any other device or combination of devices that may be used to administer food, formula, medication, hydration, fluidics, and more via enteral or parenteral nutrition to a patient. It is understood that any of these types of products may be administered with the assistance of the present invention and the term “feeds” or “feed” may be used herein to describe these products (i.e. the term “feed” does not refer only to food products, but to any product being administered with assistance of the present invention, including, for example, saline and other fluids typically administered via IV). As such, it may be said that the present invention assists with the administration of feeds from a “feeding assembly”—which is the term used for any device or equipment that is useful for administering any feed product, not just for food products. Likewise, the term “feeding” does not necessarily refer to food products, but refers to the administration of any feed products via any feeding assembly. While it is understood that any feed product and any feeding assembly can be used with the device of the present invention, certain examples herein (such as the use of a bolus tube feeding assembly, for example) may be used for purposes of describing the invention. Such examples should not be construed as limiting the invention to such specific uses.

The present invention is useful for mounting the feeding assembly in the hospital, at home and on-the-go. In particular, the device 1 of the present invention is directed to an assembly comprising a flexible arm 2, extending between a mounting piece 4 and a holding piece 3, where the mounting piece 4 is configured to securely mount the device 1 to the fixed structure 6 and the holding piece 3 is configured to securely hold the feeding assembly 5 or 12 at a feeding position determined by bending the flexible arm 2, as the holding piece 3 is connected to the flexible arm 2. The fully adjustable flexible arm 2 allows the user to modify the feeding position to a specific height above the patient thereby pre-defining the feeding rate from the feeding assembly to the patient (as a result of gravity on the product contained within the bolus feeding device 5 or as a result of the pump action of the pump and bag 12 combination Preferably the device 1 has a weight of about 6.5 to 13.5 ounces, allowing for easy mobility and use, but other weights may be acceptable if desired.

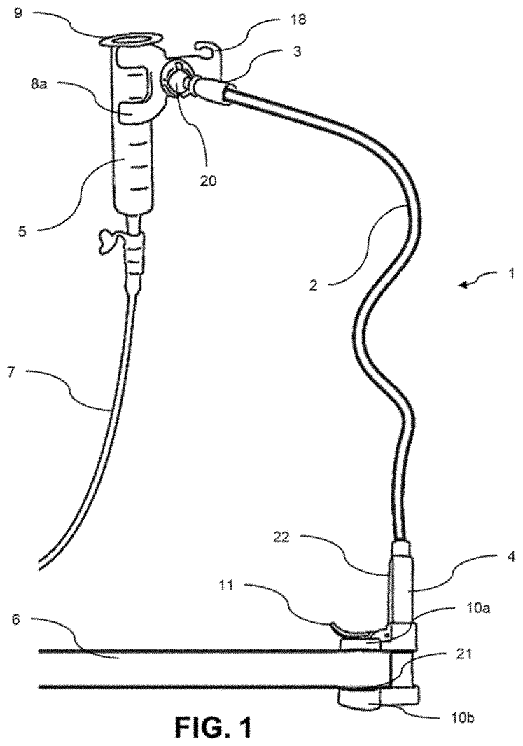

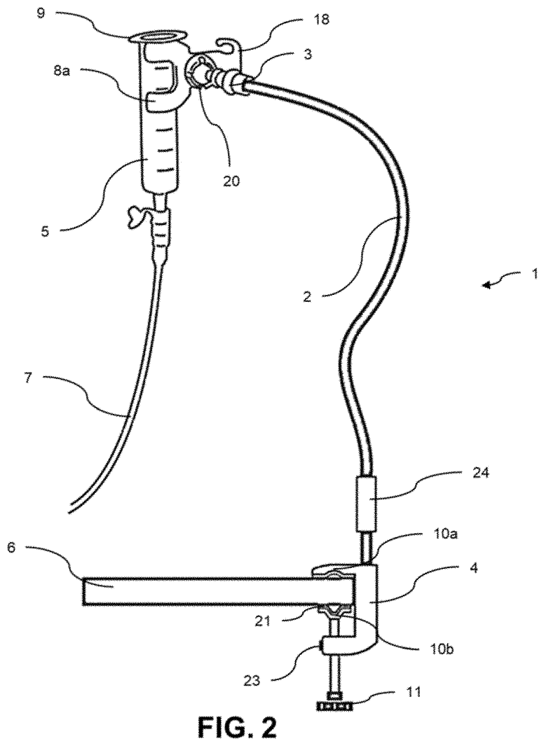

Turning now to FIG. 1, one preferred embodiment of the device 1 of the present invention may be described. As indicated above, the device 1 generally includes a holding piece 3, a mounting piece 4, and a flexible arm 2 extending between the holding piece 3 and mounting piece 4. In one embodiment, the components may be manufactured integral to one another, thereby being sold and used as a single unit. Alternatively, the components may each be manufactured separately and later assembled by the seller or end user. Manufacturing the components as separate units may offer benefits such as allowing replacement of broken parts, allowing for disassembly for disinfecting or other cleaning, or other benefits. Furthermore, as indicated above, the device is intended to be used for securely holding and mounting a feeding assembly, which may, for example, be a bolus feeding device 5 (typically a 60 to 140 mL syringe, as shown in FIGS. 1-2, but other sizes may also be used). Therefore, in one embodiment, the device may further comprise a syringe 5 manufactured integral to the device, whereby the syringe 5 is permanently attached to the holding piece 3 of the device 1. However, it is preferred that the syringe 5 be manufactured separately (despite whether the remaining components of the device 1 are integral or separate) to allow for appropriate cleaning and replacement of feeding syringes 5. Furthermore, manufacture as individual components configured to be assembled allows for easily disassembly for travel and storage (the assembly may fit into a small bag of individual components) and for cleaning.

Whether manufactured as an integral piece of the assembly 1 or a separate piece to be attached and detached from the device 1, the feeding syringe 5 must be securely held by the device 1 to allow for proper use of the device in feeding patients; however, the device can be used for administration of bolus feeding for a patient of any age. Given the nature of the use of the device 1, the holding piece 3 must be a strong, durable clip 3 having sufficient gripping strength to securely hold the feeding syringe 5 (typically a 60 mL syringe 5). In the preferred embodiment, the holding piece 3 is a spring-biased clip 3 having an open position and a closed position. The open position of the holding piece 3 is characterized as the position at which force is applied to the handles 16 of the holding piece 3 such that the jaws 8a, 8b of the holding piece 3 separate (due to compression of the spring component 17 of the holding piece) thereby allowing the feeding syringe 5 to be inserted between the jaws 8a, 8b of the holding piece 3. Of course, the closed position, is characterized as a position where force is released from the handles 16 (such that the spring 17 decompresses) thereby allowing the jaws 8a, 8b of the holding piece 3 to move closer to one another such that the jaws 8a, 8b form around the feeding syringe 5 with sufficient force to hold the feeding syringe 5 in its position between the jaws 8a, 8b of the holding piece 3. The closing force asserted on the holding piece 3 by the spring 17 is an important aspect of the invention, as it provides a secure hold on the feeding syringe 5.

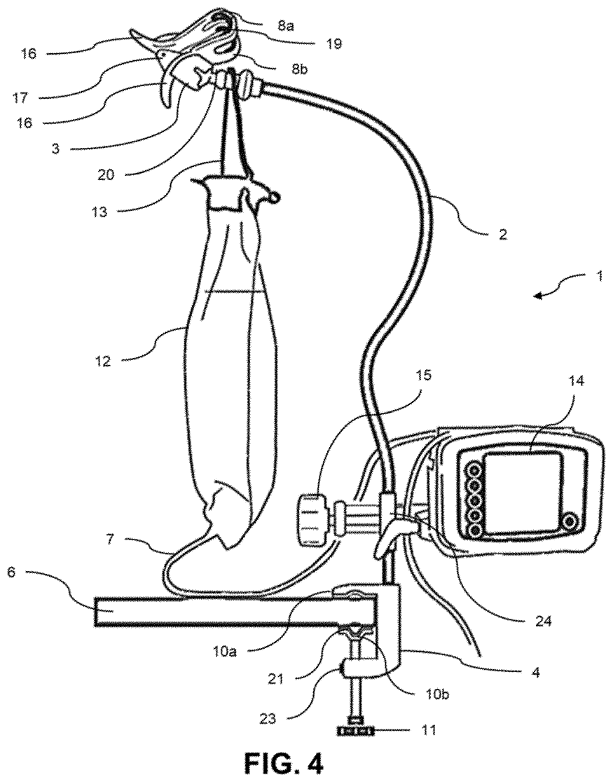

In one embodiment, the holding piece 3 is adjustable such that the size of the space between the jaws 8a, 8b in the open position (and the formation of the jaws 8a, 8b around the syringe 5 when in the closed position) can be modified to securely fit feeding syringes 5 of various sizes. For example, the preferred embodiment the holding piece 3 is capable of holding syringes 5 ranging from 1 inch in width to up to 3 inch in width or larger. In one embodiment, a hook 18 may be positioned at the holding piece 3 such that the feeding tube 7 (which extends from the feeding syringe 5 to the patient) may be held in place when the device is not in use. The holding piece 3 is preferably made from a hard, durable material (such as plastic) and is preferably lined with a nonslip material 19 (as shown, for example, in FIG. 4) on the inner surface (such as silicone, a rubber foam, or other durable nonslip material 19) to facilitate a strong hold against the feeding syringe 5. Once the syringe 5 is secured inside the holding piece 3, feeds may be added to the syringe 5 (directly by the user, caregiver, or through a pump tube 7 with one end resting in the syringe to allow venting) and administration may begin. Alternatively, feeds may be added to the syringe 5 prior to mounting the syringe 5 in the holding piece 3.

In order to allow the user to adjust the height at which the feeding syringe 5 is positioned above the patient (and therefore modify the rate at which product is administered from the feeding syringe 5 to the patient—the height and position and corresponding feed rate may, for example, be directly prescribed by the physician), the device of the present invention 1 includes a flexible arm 2 attached to the holding piece 3 (which securely holds the feeding syringe 5 as described above). In the preferred embodiment, the flexible arm 2 is a gooseneck arm 2 having sufficient flexibility to allow the user to easily modify the shape of the arm by bending the arm by hand such that as the arm 2 is bent, the positioning of the feeding assembly relative to the patient may be increased or decreased. As the arm 2 is bent, for example, the feeding syringe 5 (held by the holding piece 3 attached to one end of the flexible arm 2) may extend closer to the patient in a lower feeding position. Similarly, if the flexible arm 2 is unbent (or stretched back to a longer length), the feeding syringe 5 may be moved further away from the patient and into a higher feeding position. The terms “lower feeding position” and “higher feeding position” refer generally to the relative positioning of the feeding syringe 5 to the patient, generally where different feeding positions may result in different feed rate of product from the syringe 5 to the patient. In any event, the flexible arm 2 is configured to be manipulated by the user such that the positioning of the feeding device 1 relative to the patient may be modified as needed. It may be seen, then, that the device 1 may be adjusted without requiring tools or extreme force.

In the preferred embodiment, the flexible arm 2 is approximately 21 inch long to allow for sufficient adjustment of the device 1 to the appropriate height, but longer or shorter arms 2 may be used as appropriate. Furthermore, the flexible arm 2 preferably has a ball joint 20 (or swivel ball head) at the end intended to attached to the holding piece 3, thereby allowing the holding piece 3 to be rotated 360 degrees to further facilitate in appropriate placement of the feeding syringe 5 during administration by the device 1. The ability to rotate the holding piece 3 further allows the user to select an appropriate angle of the feeding syringe 5, as required by different feeding situations. As indicated previously, while the holding piece 3 and flexible arm 2 may be manufactured as a single unit, it is preferable that the holding piece 3 be removably attached to the flexible arm 2 so that the pieces may be disinfected between uses.

Furthermore, while the flexible arm 2 is flexible, it is also sufficiently rigid such that bending or movement of the arm is facilitated only by human force and not by gravity. For example, it would frustrate the administration of feeding and the purpose of the device if after the feeding assembly 5. 12 were attached to the device, the weight of the feeding assembly could cause the arm 2 to bend, thereby changing the feeding height of the feeding assembly 5, 12. Thus, the arm 2 must have sufficient rigidity to withstand the weight of the feeding assembly such that the arm 2 does not bend when the feeding assembly 5, 12 is attached to the holding piece 3. Thus, the flexible arm 2 is preferably made from a soft, but durable material (such as a soft plastic covered aluminum magnesium alloy) to allow for some rigidity while also allowing for some flexibility when manipulated. The flexible arm 2 is customizable by color. Other benefits of the flexible nature of the device 1 as facilitated by the flexible arm 2 are (a) the device 1 can easily be moved out of the way of the patient in the case of emergency access to the patient, (b) the device 1 can be easily moved toward a caregiver (and away from the patient) in order to add product to the syringe 5 or clean the syringe 5, etc., and (c) the device 1 can be moved to the side of the crib for a patient who must remain vented in case of a Nissen fundopolication, among many other benefits. In the preferred embodiment, the outer surface of the flexible arm 2 is a smooth surface with no ridges, folds, crevices, or other porous means. This is an important aspect of the present invention 1 because a smooth surface allows for more efficient cleaning and disinfecting of the flexible arm 2 between uses.

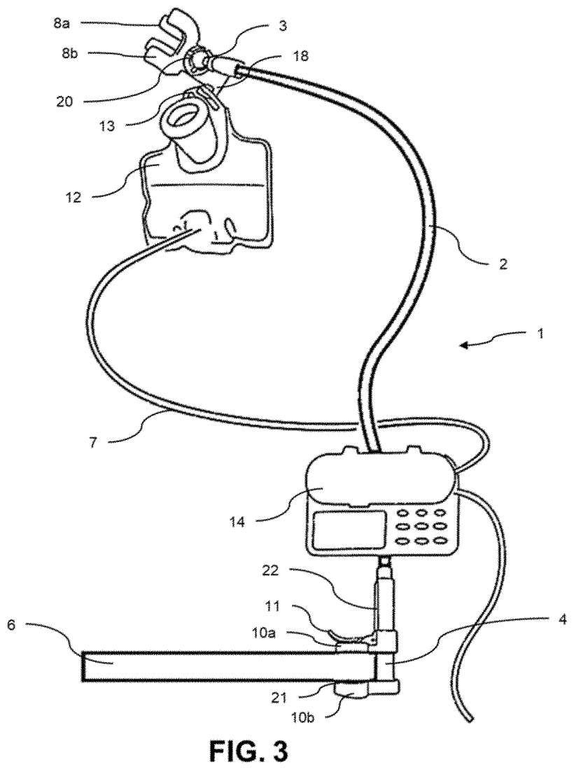

At the opposite end of the flexible arm 2 from the holding piece 3 is a mounting piece 4. Any sort of mounting piece 4 may be used, including clips or clamps. In one embodiment, the mounting piece is a 2.75 inch adjustable clamp 4 that is capable of snapping or locking into place on a variety of surfaces 6. This clamp 4 is shown in detail in FIGS. 1 and 3. The ability to lock the device 1 to a secure surface 6 using a locking clamp 4 is an important aspect of the invention 1, as a secure mount to a structure 6 is needed to avoid unnecessary injury or issues during feeding situations. The mounting piece 4 is adjustable such that the clamp 4 is capable of being closed around a number of different mounting structures (cribs, hospital beds, pack and plays, tables, strollers, wheelchairs, etc.). The clamp 4 is said to be closed when the clamp's 4 jaws 10a and 10b are moved toward each other such that the jaws 10a and 10b come into contact with a structure 6 resting between the jaws 10a and 10b and sufficient force is applied such that the clamp 4 remains mounted to the internal structure 6. The distance between the jaws 10a and 10b can be varied depending on the size of the structure 6 to which the device is being mounted. For example, the mounting piece 4 may be slightly widened to fit around a rung on an infant's crib or may be widened to fit on a dining table or side table of different thicknesses. In this regard, the adjustable nature of the mounting piece 4 allows the device 1 to be completely mobile, the user capable of unmounting the device 1 from its current mounting structure simply by opening the mounting piece 4 and remounting the device 1 on a new structure 6 by adjusting the clamp 4 to fit the new structure's 6 size. The mounting piece 4, like the holding piece 3, is preferably made from a hard, durable material (such as plastic or metal) and is preferably lined with a nonslip material 21 on the inner surface (such as silicone or rubber foam) to facilitate a strong hold against the mounting structure 6. In one embodiment, the mounting piece 4 has a sliding feature 22 in FIGS. 1 and 3 wherein a locking apparatus 11 on the clamp 4 is released allowing the jaws 10a and 10b of the clamp 4 to be moved together or apart to the appropriate thickness (corresponding to the size of the structure 6 to which the clamp 4 will be mounted). Once the clamp 4 is secured around the mounting structure 4, the locking apparatus 11 may be used to lock the clamp 4 at that position, thereby ensuring that the jaws 10a and 10b: do not separate from the mounting structure 6. Finally, when the device 1 needs to be removed from the mounting structure 6, the user simply unlocks the locking apparatus 11, slides the clamp jaws 10a and 10b away from: the mounting structure 6, and removes the device 1 from the mounting structure 6.

In another version of the device, as shown in FIGS. 2 and 4, at the opposite end of the flexible arm 2 from the holding piece 3 is a c-clamp mounting piece 4. This mounting piece 4 is preferably a 2.75 inch adjustable c-clamp that is capable of tightening into place on a variety of surfaces. The ability to tighten the device with the tightening screw 11 to a secure flat or rounded surface 6 using a c-clamp mounting structure 4 is an important aspect of the invention, as a secure mount to a structure 6 continues to be needed to avoid unnecessary injury or issues during feeding situations. The mounting piece 4 is adjustable such that the c-clamp mounting structure 4 is capable of being closed around a number of different mounting structures (cribs, hospital beds, pack and plays, tables, strollers, wheelchairs, etc.). In one embodiment, the c-clamp includes a quick release button 23 for easier adjustability of the clamp. As with the clamp shown in FIGS. 1 and 3, this c-clamp is said to be in the closed position when the c-clamp's v-plate and flat top 10a and 10b are moved toward each other by turning the tightening screw locking pieces 11 such that the v-plate and the flat top 10a and 10b come into contact with a structure 6 resting between the two plates 10a and 10b and sufficient force is applied such that the clamp mounting structure 4 remains mounted to the fixed structure 6. This allows the clamp 4 to be modified to fit around structures 6 of varying sizes. For example, the mounting piece 4 may be slightly widened to fit around a tube on a wheelchair or may be widened to fit on a flat surface of a dining table or side table of different thicknesses. In this regard, the adjustable nature of the mounting piece 4 allows the device 1 to continue to be completely mobile, the user capable of unmounting the device 1 from its current mounting structure 6 simply by untightening the tightening screw 11 and remounting the device 1 on a new structure by adjusting the c-clamp 4 to fit the new structure's 6 size. The mounting piece 4 is preferably made from a hard, durable material (such as metal) and is preferably lined with a nonslip material 21 on the inner surface (such as silicone or rubber foam) to facilitate a strong hold against the mounting structure 6.

While the invention 1 has been described herein with reference to the preferred application of holding a bolus feeding device 5, the invention 1 is not so limited and the device 1 may be used for many other applications with few to no modifications to the device 1 and only slight modifications to the method of use. For example, the device of the present invention 1 may be used to hold an assortment of pumps 14 for feeding and other uses, as shown in FIGS. 3 and 4. The pump 14 is attachable near the bottom end of the flexible arm 2 (for example, it may be attached to the hard plastic of the clip 4 at the base of the flexible arm. Alternatively, the pump 14 is attachable to any other position along the flexible gooseneck arm 2. A flat, rubber cylinder 24 (as shown, in FIGS. 2 and 4, for example) is positioned around the bottom end of the flexible arm 2. This cylinder 24 provides a place for attachment of the pump clamp such that the pump clamp does not damage the flexible arm 2. The cylinder 24 also services to add diameter to the flexible arm 2 near the bottom of the flexible arm 2, introducing some additional rigidity to the flexible arm 2. A feed bag 12 (or bag for any other material such as medicine or fluids) is hung from the holding piece 3 hook 13 (as shown in FIG. 3) or to the base of the ball joint 20 (as shown in FIG. 4), and connected to the pump 14, allowing for a fully mobile mounting apparatus 1 for various applications (for example, a patient may use the device for an exceptionally portable IV fluids mounting device). The bag is hung on the hook 18 or the base of the ball joint 20 from the feedbag strap 13. The pump and bag 14 and 12 may be attached to the device 1 with the flexible arm 2 in a fully extended position or in a curved/bent position.

Another application with few to no modifications to the device 1 and only slight modifications to the method of use is using the device 1 to hold a suction tubing or other dentistry and medical aids. The suction may be held by the clip 3 on the device 1 with the flexible arm 2 in a fully extended position or in a curved/bent position.

Unless otherwise stated, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein. It will be apparent to those skilled in the art that many more modifications are possible without departing from the inventive concepts herein.

All terms used herein should be interpreted in the broadest possible manner consistent with the context. In particular, the terms “comprises”' and “comprising” should be interpreted as referring to elements, components, or steps in a non-exclusive manner, indicating that the referenced elements, components, or steps may be present, or utilized, or combined with other elements, components, or steps that are not expressly referenced. When a Markush group or other grouping is used herein, all individual members of the group and all combinations and subcombinations possible of the group are intended to be individually included. All references cited herein are hereby incorporated by reference to the extent that there is no inconsistency with the disclosure of this specification. When a range is stated herein, the range is intended to include all sub-ranges within the range, as well as allindividual points within the range. When “about,” “approximately,” or like terms are used herein, they are intended to include amounts, measurements, or the like that do not depart significantly from the expressly stated amount, measurement, or the like, such that the stated purpose of the apparatus or process is not lost.

The present invention has been described with reference to certain preferred and alternative embodiments that are intended to be exemplary only and not limiting to the full scope of the present invention.

We claim:

1. A method for administering feed product from a feeding apparatus to a patient, the method comprising the steps of:
a. assembling a device comprising:
i. a flexible arm comprising a first end, a second end, and an arm distance positioned therebetween;
ii. a holding piece connected to the first end of the flexible arm, wherein the holding piece comprises a set of jaws;
iii. a mounting piece connected to the second end of the flexible arm, wherein the mounting piece comprises a set of jaws;
b. positioning the jaws of the mounting piece around a fixed surface;
c. closing the jaws of the mounting piece around the fixed surface, thereby mounting the device to the fixed surface;
d. attaching the feeding apparatus to the holding piece; and
e. selectively bending the flexible arm to a desired position, thereby moving the holding piece to a desired holding position, thereby moving the feeding apparatus to a desired feeding position.

2. The method of claim 1, wherein the feeding apparatus comprises one of a bolus syringe and a bottle.

3. The method of claim 2, wherein the step of attaching the feeding apparatus to the holding piece comprises the steps of:
a. opening the jaws of the holding piece;
b. positioning the jaws of the holding piece around the feeding apparatus; and
c. closing the jaws of the holding piece such that the jaws exert a force on the feeding apparatus, thereby securely holding the feeding apparatus within the holding piece.

4. The method of claim 1, wherein the holding piece further comprises a hook.

5. The method of claim 4, wherein the feeding apparatus comprises a bag having a strap.

6. The method of claim 5, wherein the step of attaching the feeding apparatus to the holding piece comprises the step of hanging the strap of the bag from the hook of the holding piece, thereby securing the bag to the device.

7. The method of claim 1, wherein the feed product is one of food, IV fluids, saline, and medication.

8. A device useful in the administration of feed product from a feeding apparatus to a patient, the device comprising:
a. a flexible arm comprising a first end, a second end, and an arm distance positioned therebetween;
b. a holding piece connected to the first end of the flexible arm, wherein the holding piece comprises a set of jaws, further wherein the holding piece is configured to hold the feeding apparatus;
c. a mounting piece connected to the second end of the flexible arm, wherein the mounting piece comprises a set of jaws, further wherein the set of jaws of the mounting piece is configured to be positioned around a fixed structure, and further wherein the mounting piece is configured to be closed such that the jaws of the mounting piece exert a force on the fixed structure, thereby affixing the device to the fixed structure,
wherein the flexible arm is configured to be bent such that the holding piece connected to the first end of the flexible arm is moved to a desired holding position, thereby positioning the feeding apparatus held by the holding piece at a desired feeding position.

9. The device of claim 8, wherein the feeding apparatus comprises one of a bolus syringe and bottle.

10. The device of claim 9, wherein the set of jaws of the holding piece is configured to be positioned around the feeding apparatus and further wherein the holding piece is configured to be dosed such that the jaws of the holding piece exert a force on the feeding apparatus, thereby securing the feeding apparatus within the holding piece.

11. The device of claim 10, wherein the feeding apparatus comprises a lip, further wherein the lip of the feeding apparatus is configured to rest on the jaws of the holding piece, thereby prohibiting the feeding apparatus from sliding through the jaws of the holding piece.

12. The device of claim 8, wherein the feeding apparatus comprises a bag.

13. The device of claim 12, wherein the holding piece comprises a hook, wherein the bag is configured to hang from the hook of the holding piece, thereby securing the bag to the device.

14. The device of claim 12, comprising a connector joint connecting the holding piece and the flexible arm, wherein the bag is configured to hang from the connector joint, thereby securing the bag to the device.

15. The device of claim 12, wherein the feeding apparatus is fluidically connected to a pump, further wherein the pump is configured to attach to the flexible arm, thereby mounting the pump to the device.

16. The device of claim 8, wherein the holding piece is a butterfly clip.

17. The device of claim 8, wherein the mounting piece is a clamp.

18. The device of claim 17, wherein the mounting piece is a C-clamp.
